(19) United States
(12) Patent Application Publication
(10) Pub. No.: US 2007/0265683 A1
(43) Pub. Date: Nov. 15, 2007

(54) Method and Apparatus for Treatment of Tinnitus and Other Neurological Disorders by Brain Stimulation in the Inferior Colliculi and/or In Adjacent Areas

(76) Inventor: Dov Ehrlich, Rehovot (IL)

Correspondence Address: DARBY & DARBY P.C., P.O. BOX 770, Church Street Station, New York, NY, 10008-0770, US

(21) Appl. No.: 11/575,855

(22) PCT Filed: Sep. 22, 2005
(86) PCT No.: PCT/IL05/01017

§ 371 Date: May 17, 2007

Related U.S. Application Data
(60) Provisional application No. 60/612,530, filed on Sep. 24, 2004.

(51) Int. Cl. A61N 1/00

(52) U.S. Cl. 607/55; 600/25

(57) ABSTRACT

Electrical stimulation is applied to the inferior colliculus or colliculi (IC), in order to diminish tinnitus by revising auditory pathway neuronal activity. This intervention diminishes tinnitus and treats other neurological and otological disorders. The locations and methods of electrode placement and anchoring and the structure of the electrodes are an advance over prior treatments. Other treatment locations in the nearby region of the IC, including the superior colliculi (SC) and Peri-aqueductal gray (PAG), provide treatments for other disorders and symptoms such as partial hearing loss and pain. The IC is a unique choice for the treatment of tinnitus and other disorders because an electrode placed in that region enables minimal invasiveness. The anchoring location also uniquely minimizes invasiveness by providing the option of residing in the meninges instead of the brain tissue. The shape of the electrode and its anchoring process uniquely match the brain's anatomy in order to provide greater specificity in diagnosis and treatment. Stimulation of areas near the IC, particularly the superior colliculus and peri-aqueductal gray, can be used to treat various neurological disorders. Customized feedback from the implantable system enables the creation of customized treatment programs.

### FIELD AND BACKGROUND OF THE INVENTION

The present invention relates to implantable stimulation systems and methods, and more particularly relates to an implantable stimulation system and method utilizing one or more electrodes, anchored in an improved manner, that are implanted at the inferior colliculus and/or adjacent areas, in order to treat tinnitus and other relevant neurological and otological disorders. The treatment is fully controlled and reversible.

Tinnitus: Overview and Prevalence

Tinnitus is the occurrence of an auditory sensation without the presence of an acoustic stimulus. Tinnitus is frequently associated with a loss of peripheral (meaning “outside the brain”) auditory sensitivity, and can occur with normal cochlear function as well as after deafferenation (that is, disconnection of incoming sensory tracts in the brain). Tinnitus is experienced chronically by many. It is estimated that tinnitus affects 1 out of every 200 adults. Tinnitus severity increases with age and greatly impairs the individual's quality of life.

Approximately 50 million Americans suffer from some degree of tinnitus. Of these, about 12 million have tinnitus severe enough to seek medical attention; and about 2 million patients are so seriously debilitated that they cannot function on a “normal,” day-to-day basis.

Tinnitus severity increases with age and greatly impairs the individual's quality of life.

Background Anatomy

A discussion of the anatomy of the auditory system is necessary to understand properly the central role of the inferior colliculus and the innovation of the current invention.

The sensory systems in the central nervous system (CNS) include an ascending tract that takes the sensory data from its point of entrance to the relevant part of the brain that processes it. In addition, these systems also have a descending tract, originating in the cortex. At the point that ascending and descending tracts meet, the descending tract exerts its modulating effect on the incoming data according to the needs of the brain.

The Ascending System

Auditory data is generated in the inner ear, from which it is transmitted up to the brain via the 8^{th} cranial nerve. Ninety five percent of the fibers in this nerve emanate from inner hair cells that conduct auditory data, and five percent represent outer hair cells, whose activity can change the mechanical properties of the inner hair cells. When the source of tinnitus is in the inner ear, activating the outer hair cells can suppress tinnitus by affecting their sound conduction properties.

Upon entrance into the brainstem, each neuronal fiber splits into three branches that synapse in three parts of each cochlear nucleus in the pons. This branching is the basis for simultaneous parallel processing of the auditory data. The two cochlear nuclei are interconnected. Most of the post-synaptic fibers from the nuclei decussate, and form the contralateral lateral leminiscus (LL); some do not decussate, however, and ascend in the ipsilateral LL. (“Contralateral” refers to the opposite side of the brain, and “ipsilateral” to the same side of the brain.) Right and left LL axons terminate on the corresponding inferior collicullus (IC) to form the second synapse of the ascending auditory pathway.

All auditory fibers synapse in the IC. The right and left ICs are extensively interconnected. The IC receives data from non-auditory parts of the brain so auditory data in this site may be modulated by non-auditory systems. Further, the IC is described as the auditory reflex center, mediating the motor responses that are evoked automatically in response to auditory stimuli, such as the immediate turning of the head towards a sudden loud noise.

The post-synaptic fibers proceed to the medial geniculate body (MGB) of the thalamus for the third synapse. There are about 250,000 neurons in this pathway—a fact which provides strong evidence for the parallel processing of the auditory data. Here an interaction with other thalamic nuclei further modulates the incoming data. The thalamic neurons connect to the auditory cortex.

Scheme of the Auditory Ascending Pathways

FIGS. 1 and 2 show a schematic representation of the auditory ascending pathways, showing data entering via the cochlear nerve, decussating, ascending to the IC, where it synapses, and then ascending to the thalamus and cortex.

Like other sensory modalities, the ascending auditory fibers have a topographical order; they are located according to the sound frequency that they mediate. This is called tonotopic arrangement.

The two inferior colliculi are integrally involved in hearing. They relay information to the MGB of the thalamus. The IC is predominantly concerned with detecting and analyzing auditory stimuli. The IC responds to sounds arriving from either ear; thus, the IC analyzes and localizes the source and direction of various sounds.

Anatomy and Physiology of the Descending System

Descending auditory pathways travel in parallel to the ascending ones, and inhibit the ascending auditory data. Descending pathways may filter out the irrelevant auditory data, thereby helping to extract useful data from the auditory noise. In the first descending pathway, the olivo-cochlear bundle (OCB) connects the olivary nucleus in the brainstem to the hair cells in the cochlea. The second descending pathway extends from the primary auditory cortex to the thalamus and the inferior colliculus. Sound evokes activity in the fibers of the OCB and electrical stimulation of this bundle can affect the activity in auditory nerve fibers. Activity in OCB can affect properties of the outer hair cells in the cochlea, which, in turn, affects activity in ascending auditory nerves. Damage to the second system (from the cortex to the IC) changes the frequency tuning of cells in the MGB and IC.

The “Non-Classical” Auditory System

The auditory system also has a “non-classical” set of tracts, transmitting less processed auditory data. This system is much less studied than the classical system described above. This system receives data from the classical system, and transmits it centrally in parallel, together with data received from other neural systems, including the somatosensory, visual, and vestibular ones. This system also “exports” auditory data to influence other sensory systems. The non-classical system also has a descending set of fibers from the cortex to the IC and other structures.

FIG. 3 shows the descending auditory pathways.

The Superior Colliculi (SC)

The IC sends nerve fibers to the pons, medulla, the SC (a visual modulation center), the spinal cord, and the nuclei controlling the neck and facial musculature. Hence, via the IC, auditory impulses can trigger head and body turning and orientation toward sound sources.

The superior colliculi can be functionally divided into superficial and deep layers. The superficial layers receive considerable input from the retina as well as from the temporal and occipital visual cortex, and respond to moving stimuli. The superficial layers also project to vision-related cranial nerve nuclei.

By contrast, the intermediate and deeper layers receive converging motor, somesthetic, auditory, visual, and reticular input, serve as an extension of the reticular formation, and interconnect with the caudal medulla and cranial nerves associated with movement of the head and eyes.

There are only few studies on electrical stimulation of the SC. These studies demonstrate the role of the SC in eye movements and the musculature of the face and neck. Microstimulation of the rostral parts of the inner layers of the SC produces facial motor responses and activates the motor neurons of the neck muscle. One of many innovations of the current patent is to provide therapy for partial hearing loss by stimulating the SC in order to assist motor adjustment to the origination of the sound.

FIG. 4 illustrates the location and connections of the superior colliculus.

The Resemblance Between the Pain System and the Auditory System

The pain descending tract is extensively explored. The brainstem has a neuronal center, which is the origin of a descending inhibitory system, whose fibers go down to the spinal cord, and interact with incoming pain data, mostly in an inhibitory way. This brainstem center is activated by the incoming pain messages themselves, but is also under continuous influence of cortical structures such as the frontal lobbes and the limbic system.

Thus, when the body is experiencing pain, it first alerts the brain that a painful event has taken place, and then the brain acts to diminish the intensity of the pain experience, so that it can best deal with the situation, including removal of potential damage.

A commonly used example for this activity is the soldier wounded in the battlefield that does not experience pain while still fighting, as the brain realizes that it does not have the luxury of suffering from pain at the moment. Then, when evacuated and away from immediate danger, the soldier experiences pain. This descending system is utilized medically, when a spinal cord stimulator is implanted in patients suffering from chronic pain. This method works by activation of the descending inhibitory pain pathways.

The descending auditory tract inhibits tinnitus in a manner parallet to the descending inhibition of pain. The tract that goes from the thalamus down to the brainstem is likely the site of entry of the auditory data; en route, nearly all descending fibers synapse in the IC. Thus, IC seems to be a major modulation point for the processing of incoming auditory data. As such, it is an ideal site for external intervention in auditory activity.

In this patent, all references to the inferior collicular region include the PAG and SC, which are sufficiently close to be stimulated by electrodes in the IC region, as shown in FIG. 5.

Similarities Between Severe Tinnitus and Chronic Pain

The symptoms and signs of severe tinnitus and chronic pain have many similarities:

Individuals with chronic pain perceive normal stimulation of the skin to be painful (allodynia). The “wind up” phenomenon that occurs in severe pain is the worsening of pain sensation from repeated stimulation. In tinnitus, the painful or unpleasant sensation of sound that subjects with severe tinnitus experience resembles allodynia. Repeated sound stimulation causes an increased painful sensation that may be analogous to the “wind-up” phenomenon.

Emotional and systemic reactions: both chronic pain and severe tinnitus are often associated with reactions such as anxiety, nausea, and general stress reaction such as elevated blood pressure.

Similarities in the hypotheses about the generation of pain and tinnitus: Although less severe tinnitus may be generated in the ear, it is believed that severe tinnitus is caused by changes in the nervous system. Similarly, acute pain is the result of local tissue injury, but chronic pain is generated in the CNS.

Neural mechanisms of severe tinnitus and chronic pain: Both conditions probably result from reorganization of the CNS. The current hypotheses about chronic pain focus on wide dynamic range neurons (WDR) that normally mediate tone and vibration but change their responses to such stimuli. It is believed that WDR neurons' excitability increases if excitatory input increases or inhibitory input decreases. Less is known about tinnitus. However, it is known that noise exposure can cause development of hyperexcitability of neurons in the IC by decreasing GABA inhibition. The IC has a role in chronic tinnitus that is similar to WDR neurons in chronic pain.

Therefore, since the IC is very close to the peri-aqueductal gray (PAG), a modulation station for pain data and thus a site for control of pain, activation of the IC-adjacent area of the PAG may suppress pain as well.

Deep Brain Stimulation (DBS)

DBS is a surgical procedure used to treat a variety of disabling neurological symptoms such as those of Parkinson's disease (PD), essential tremor (ET), intractable epilepsy, refractory cluster headache, and psychiatric conditions such as obsessive compulsive disorders.

After receiving FDA approval for essential tremor (1997) and PD (2002), DBS received a Humanitarian Device Exemption (HDE) for treating dystonia. The benefit of this treatment is also being investigated for several other disorders, including but not limited to pain, and depression.

DBS uses a surgically implanted, battery-operated medical device called a neuro-stimulator, similar to a heart pacemaker and about the size of a stopwatch, to deliver electrical stimulation to targeted areas deep in the brain that are key relay sites in the control and regulation of movement, seizures, and emotional/motivational behaviors. Stimulation of these small anatomic sites influences physiologic activity in a more widespread area of the cortex, leading to the desired beneficial response.

The Surgery

There are many approaches as to how the surgery is performed. Generally, the surgery starts by applying a stereotactic frame around the head to facilitate the identification of the precise target in the brain.

With an MRI or CT scan, a temporary microelectrode is inserted into the brain through a small opening in the skull. Before the surgeon makes the small opening, local anesthetic is administered, and the patient is awake. The patient does not experience any pain because brain tissue does not generate pain signals. All surgeons perform intraoperative stimulation to test for efficacy and confirm a lack of side effects. Once the target is found, the microelectrode is removed and the permanent implant device, containing at least one electrode, is placed in the cranial cavity. (The present invention emphasizes the advantage of placing this device substantially on the brain surface, ideally 100% external to the surface, but it may be less. We define the surface of brain as substantially having an outside plane perpendicular to a theoretical line entering the interior of the brain or its bulges and depressions, so that in the ideal embodiment, the implant device containing at least one electrode will be planar to the surface.) This portion of the surgery takes nearly the whole day. The electrodes are then covered up and the incisions closed. Postoperative imaging is used to confirm appropriate target localization. The patient usually recuperates in the hospital for approximately three days and is then discharged.

The second stage of the surgery occurs approximately one week later. Surgeons typically use general anesthesia or sedation during this procedure. The surgeon makes a small incision in the subclavicular area and creates a pocket. The neuro-stimulator is then placed in the pocket. The leads from the electrode are tunneled under the scalp, under the skin of the neck, and down to the pocket. This procedure takes several hours, and the patient is discharged the next day. The stimulators are turned on for the first time within a few weeks after implantation. Adjustment of medication, as well as a series of adjustments in the electrical pulse, are made during the following weeks or months.

Electrical Stimulation for the Treatment of Tinnitus

Tinnitus is poorly controlled by medications and by other interventions (surgery, cochlear implant, hearing aids, maskers). None of these current treatments for tinnitus have proved consistently effective in well-designed clinical trials involving large patient numbers.

Based on the vast experience of the DBS methodology for a large variety of disabling disorders described below, electrical stimulation of the IC for the treatment of severe tinnitus, a major innovation of this patent, may be the most effective treatment option.

The rational for electrical stimulation of the IC for debilitating tinnitus relies on its key role in auditory regulation and processing.

The IC, in contrast to the GPi (Globus pallidus) and STN (subthalamic nucleus), is more distinct, small, and superficially located, and hence the penetration will not go through much brain tissue as in other DBS procedures. Thus, electrical stimulation of the IC will effectively influence the auditory system without affecting other systems, and diminish tinnitus and treat other neurological disorders.

Therefore, the present invention seeks to provide an improved treatment for this disease by targeting the inferior colliculi, and by doing so in a method that improves on current electrode techniques for brain stimulation.

FIG. 6 illustrates the implantable components of a DBS system. The system consists of three parts: the electrode, the pulse generator, and a wire or wireless connection between them. The location of the electrode is merely for demonstration.

Electrode Construction

There are many types of electrodes in the marketplace. Our differentiation stems from the size and shape of our electrode that matches the auditory areas in the inferior colliculus, and the adjacent region of the IC to cover the SC and PAG. The implant device, further, is ideally an electrode array with multitude of stimulation points that analyzes and records the distribution of frequencies in the IC region. The stimulation parameters will likely be unique to each anatomic region stimulated, particularly in the IC, which has a somatotropic arrangement.

There are two options to its configurations. (1) The electrode can be located on the surface of the IC. It will be placed beyond through the two outer meninges, the dura and the arachnoid, and positioned on top of the pia, which is the innermost layer of the meninges on the surface of the brain. (2) Alternatively the electrode can be needle like in shape, optionally with a fan to open the implant device.

The said electrode is equipped with anodal blocking to limit the effect of the electrode only to the site of stimulation and prevent leakage of current along the auditory tracts.

Anchoring Mechanisms

The electrode is fixated to the best site for electrical stimulation. Most anchoring occurs by proliferation of scar tissue around the electrode, but the electrode in our invention may also be fixed to the skull or dura including the tentorium with a temporary or permanent holder. Another possibility unique to our invention is a fixation to the pia mater.

The Pulse Generator

This device may include the internal feedback feature discussed below.

Stimulation Patterns

Stimulation patterns can take many forms, such as sinus waves, square waves, bursts with intervals and constant parameters, and bursts with changing parameters such as different frequencies in different bursts. The changes are meant to minimize adaptation to the pattern, and maintain the effect of stimulation over a long period of time.

### PRIOR ART

Prior art has not described the use of Deep Brain Stimulation in the IC region for tinnitus.

WO 03/035168 to Gibson et al., describes an electrode array that is implantable within the inferior colliculus of the midbrain and/or other appropriate regions of the brain of an implantee and adapted to provide electrical stimulation thereto.

The objective of the invention, as stated, is to provide a hearing sensation to persons with hearing loss. It does not mention tinnitus. Furthermore, the shape and placement of the electrode differ from the current invention.

U.S. Pat. No. 6,456,886 and U.S. Pat. No. 5,697,975 to Howard et al., describes a neural prosthetic device for reducing or eliminating the effects of tinnitus, which is inserted into a tinnitus patient's primary auditory cortex (or thalamus). The prosthetic device includes a stimulation device for outputting processed electrical signals and an electrode arranged in the primary auditory cortex having a plurality of electrical contacts. Each of the plurality of electrical contacts independently outputs electrical discharges in accordance with the electrical signals. In another embodiment, a catheter is inserted into the tinnitus patient's primary auditory cortex or thalamus. The catheter microinfuses drugs which suppress or eliminate abnormal neural activity into disperse geometric locations in the cortex or thalamus, thereby reducing or eliminating the effects of the patient's tinnitus.

The above patent involves placement of a prosthetic device in the cortex or thalamus and does not mention the IC. In addition, it is highly invasive, has the potential to destroy brain tissue there and provide a focus for the development of seizures. It could generate more noise by activating neurons of the auditory cortex. The likelihood of interference with normal hearing seems to be high in this type of stimulation. Further, the two auditory cortices do not communicate readily with each other, and it is likely that tinnitus will not be eliminated by this stimulus. The ICs are at a much lower level, and are very well connected with each other, so stimulation of the IC is more likely to be effective.

U.S. Pat. No. 5,735,885 to Howard et al., describes a method for implanting a neural prosthetic device into a target zone of a patient's brain for reducing or eliminating the effects of tinnitus. The prosthetic includes a stimulation device for outputting processed electrical signals and an electrode, which is arranged in the target zone having a plurality of electrical contacts.

The above patent does not mention the IC. In addition, it is highly invasive and has the potential to destroy brain tissue there and provide a focus for the development of seizures.

U.S. Pat. No. 6,649,621 to Kopke et al., describes methods for preventing and treating sensorineural hearing loss and is directed to the restoration or protection of hair cells in individuals experiencing a non-presbycusis type sensorineural hearing loss or who are at risk for an acute hearing loss due to exposure to noise, toxins, or other stressors. More specifically, this invention relates to the use of agents which augment inner ear antioxidant defenses to prevent and/or reverse hearing loss induced by noise, toxins, or other stressors.

The above patent does not address the current invention although on pages 10 and onwards (Example 6), there is described an electrode implanted at the inferior colliculus but without a method for treating tinnitus. The above patent discloses an intention for the purpose of gathering information, and not for a treatment method.

U.S. Pat. No. 5,667,514 to Heller describes an inserting device for inserting an elongated thin flexible surgical member, such as an electrode, and the like, into body tissues.

In Heller's patent, lines 11-13 on page 5 read, “The device described above may be used, for example, to stereotactically insert an electrode array into the auditory cortex or inferior colliculus to provide auditory stimulation. An electrode array with geometry similar to a cochlear implant electrode may be used for this purpose.” This patent however does not specifically address the current invention and does not mention any disease treatment method.

The following patents are mentioned as being of remote relevance: U.S. Pat. No. 5,545,219 to Kuzma; U.S. Pat. No. 6,671,559 to Goldsmith et al; US 2004/0133250 to Ball et al.; U.S. Pat. No. 6,032,074 to Collins; U.S. Pat. No. 6,656,172 to Hildebrand; WO 02/080817 to Gibson et al.; U.S. Pat. No. 6,301,492; U.S. Pat. No. 6,631,295; U.S. Pat. No. 5,716,377; U.S. Pat. No. 5,938,688; U.S. Pat. No. 6,301,492.

There is ample scientific literature supporting the significance of the IC in severe tinnitus and other neurological disorders, but none of it suggests stimulating the IC electrically. Indications for effectiveness of the current invention are supported by the following selected bibliography.

### SELECTED BIBLIOGRAPHY

Aouizerate B, Cuny E, Martin-Guehl C, Guehl D, Amieva H, Benazzouz A, Fabrigoule C, Allard M, Rougier A, Bioulac B, Tignol J, Burbaud P. Deep brain stimulation of the ventral caudate nucleus in the treatment of obsessive-compulsive disorder and major depression. Case report. J Neurosurg. 2004;101(4):682-6.
Arnold W, Bartenstein P, Oestreicher E, Romer W, Schwaiger M. Focal metabolic activation in the predominant left auditory cortex in patients suffering from tinnitus: a PET study with [18F]deoxyglucose. ORL J Otorhinolaryngol Relat Spec. 1996; 58(4):195-9.
Axelsson A, Ringdahl A. Tinnitus—a study of its prevalence and characteristics. Br J Audiol 1989; 23(1): 53-62.
Bauer C A, Brozoski T J, Holder T M, Caspary D M. Effects of chronic salicylate on GABAergic activity in rat inferior colliculus. Hear Res 2000; 147: 175-182.
Berk C, Carr J, Sinden M, Martzke J, Honey C R. Assessing tremor reduction and quality of life following thalamic deep brain stimulation for the treatment of tremor in multiple sclerosis. J Neurol Neurosurg Psychiatry. 2004; 75(8): 1210-11.
Brandao M L, Melo L L, Cardoso S H Mechanisms of defense in the inferior colliculus. Behav Brain Res. 1993 20; 58(1-2): 49-55
Casseday J H, Ehrlich D, Covey E. Neural mechanism of sound duration; control by excitatory-inhibitory interactions in the inferior colliculus. J Neurophysiol 2000; 84: 1475-87.
Coles R R A. Epidemiology of tinnitus. I. Prevalence. J Laryngol Otol 1984; 9 Suppl; 7-15
Eggermont J J. On the pathophysiology of tinnitus; a review and a peripheral model. Hear Res 1990; 48: 111-123.
Faingold C L, Gehlbach G, Caspary D M. On the role of GABA as an inhibitory neurotarnsmitter in inferior colliculus neurons: iontophoretic studies. Brain Res 1989; 500: 302-12.
Gerken G M. Central tinnitus and lateral inhibition: an auditory brainstem model. Hear Res 1996; 97: 75-83.
Huffman R F, Henson O W. The descending auditory pathway and acousticomotor systems: connections with the inferior colliculus. Brain Res Brain Res Rev. 1990; 15(3): 295-323.
Jastreboff P J. Phentom auditory perception (tinnitus); mechanism of generation and perception. Neurosci Res 1990; 8: 221-254.
Lockwood A H, Salvi R J, Coad M L, Towsley M L, Wack D S, Murphy B W. The functional neuroanatomy of tinnitus: evidence for limbic system links and neural plasticity. Neurology. 1998; 50(1):114-20.

There is thus a widely recognized need for, and it would be highly advantageous to use, an implanted electrode system at the IC and/or adjacent to it for the treatment of tinnitus and other neurological disorders.

### SUMMARY OF THE INVENTION

Detailed Description of the Surgery:

1. The electrode is inserted through a burr hole into the cranial cavity. This can be done in several ways.
An optional microelectrode for determining the exact position in the brain.
After location of the brain structures, the initial microelectrode can be removed.
An IC region implant device and/or a needle-like implant device that can be inserted like a needle, but when approaching the surface, can, at command, be opened to a fan like structure, that will land on the IC surface.
The electrode is directed to reach the IC using a stereotactic or equivalent system (for example, image-guided surgery). This is done by local anesthesia in the alert patient during ambulatory treatment. This sequence of surgical steps is standard, but the innovations of the current invention involve at this point the location of the implant device that targets the IC region and its method of opening.
2. The implant device is directed to the relevant side to treat tinnitus, usually the side contralateral to that of greater tinnitus. If tinnitus is bilateral, with the same intensity, it will be inserted in the left IC. If required, the electrode can be inserted to the other side.
3. Once the implant device reaches the IC, it is adjusted to start stimulation, and patient is asked whether tinnitus has diminished.
4. Stimulation is given at several loci within the IC and its adjacent areas, with usage of several stimulating points within the implant device, until the one with best effect is found.
5. The implant device is fixated to the best site, as described above, and the stimulator and power source are implanted subcutaneously. The patient can control the stimulation parameters with a remote control unit.
   Electrode
1. IC region implant device, with a shape adjusted to the external surface of the IC, and, optionally, the SC and PAG. (The IC region is defined as including the IC, SC, and PAG in this patent.)
2. The implant device has many stimulating points such that it does not invade brain tissue, but still can make various combinations of point stimulation, thereby pinpointing the focus of stimulation to a specific point or points in the IC, or in the region of the IC, to reach other desired targets such as the SC and PAG. The stimulating points are adjusted to the tonotopical arrangement of the IC. This is a process of functional imaging of the placement of the implant device.
3. The implant device is equipped with anodal blocking. An additional stimulating surface on the same implant device can be used, so that the ‘leak’ of current that might unwantedly activate varous neuronal structures—near or around the stimulated target—is blocked. With the additional stimulating surface that can be inserted either proximally or distally to the IC, the implant device can either block the neural traffic ascending from the IC towards the thalamus, or block the traffice descending from IC towards the ear. By blocking certain noises and some interference with hearing discriminability, it will be possible to limit the effect of the electrode only to the site of stimulation, and prevent ‘leakage’ of current along the auditory tracts.
4. The implant device or the stimulator will have internal feedback capability feature which analyzes the electrical activity to identify the tinnitus arid thereby adjust the stimulation's frequency, time, and space pulses to block the tinnitus.
5. The interactive modality entails the patient identifying his or her auditory experience for the system including the internal feedback feature, in order to determine various daily life sounds that are not to be touched, or even to be amplified, and for tinnitus sounds, that are to be suppressed. A library of templates and an automatic process will compare the sound to the template and classify it. Then the internal feedback component will identify the neural profiles of the wanted/unwanted auditory experiences, and intervene appropriately. This will be an ever-evolving interactive process between the patient and the machine.
6. This approach will be used for people with hearing loss as well. The feedback component will learn what are the wanted signals, and block all others, so that the patient can use all his or her hearing capacity for what he or she needs to hear—for example, human voices to be preferred over mechanical environmental sounds. This component can function as a nerve amplifier by augmenting (stimulating) the nerve activity and the wanted sounds.
   Fixation/Anchoring

Fixation can be accomplished in several ways:

fixating the electrode to the bone of the skull

an extension attached to the electrode that holds on to the tentorium

natural scarring at the level of the pia mater

using a temporary holder that can be pulled out through the burr hole after implantation

using a temporary holder that self-destructs after the tissues cause the electrode to be fixated

The implantable system will be useful for other neurological problems. Correlation of auditory hallucinations with neuronal activity may enable stimulation in the IC region that diminishes the problem. Epileptics with an auditory component to their disease will be assessed by a combination of questionnaire and electroencephalogram to determine their suitability for stimulation to block foci of epileptic activity.

The present invention successfully leverages the presently known electrode and DBS configurations by providing an innovative method specifically directed to the IC and its adjacent region, an innovative location that is less invasive and destructive than other brain targets such as the GPi and STN. The implant device apposes the surface of the brain, here defined as the surface that faces the meninges.

This patent describes both the physical apparatus of an implantable system with the innovations of location in the IC area, an anchoring arm, programmable components with a feedback system, the fan-like implant device insertion device, anodal blocking, and the shape of the implant device, and the method of inserting and using this implantable system to diagnose and treat neurological and otological disorders by appropriate brain stimulation in an interactive process with the patient.

### BRIEF DESCRIPTION OF THE DRAWINGS

The invention is herein described, by way of example only, with reference to the accompanying drawings, wherein:

FIGS. 1 and 2 show a schematic representation of the auditory ascending pathways;

FIG. 3 shows the descending auditory pathways;

FIG. 4 illustrates the location and connections of the superior colliculus;

FIG. 5 shows the PAG and adjacent IC;

FIG. 6 illustrates the implantable components of a DBS system;

FIG. 7 is a sagittal view of the midbrain including the colliculi.

FIG. 8 is a rear view of the colliculi.

FIG. 9 is a side view of the surface electrode.

FIG. 10 is a side view of an anchoring device.

FIG. 11 is a side view of a fanned, insertable, needle electrode.

FIG. 12 is a cross-sectional view of the layers of structures surrounding the brain that illustrates possible locations of electrode placement.

FIG. 13 is a flow chart of feedback steps.

FIG. 14 is a cross-section of an electrode configuration with a shape that approximates the external shape of the SC, PAG, and IC.

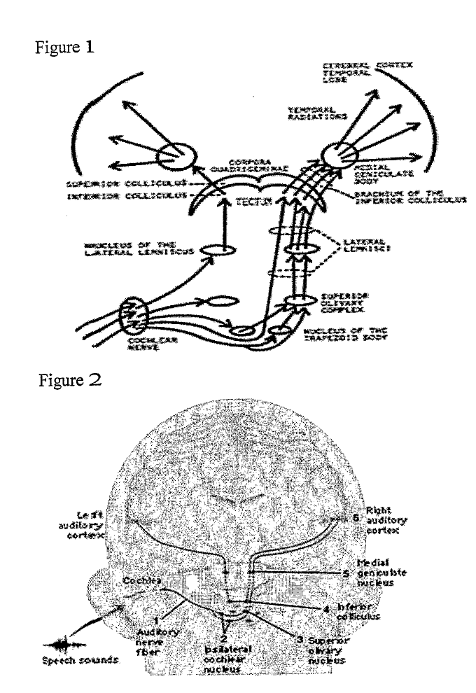

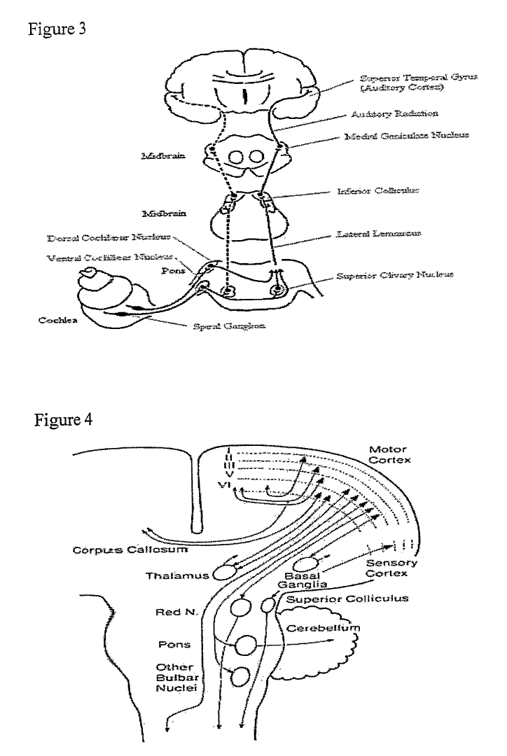

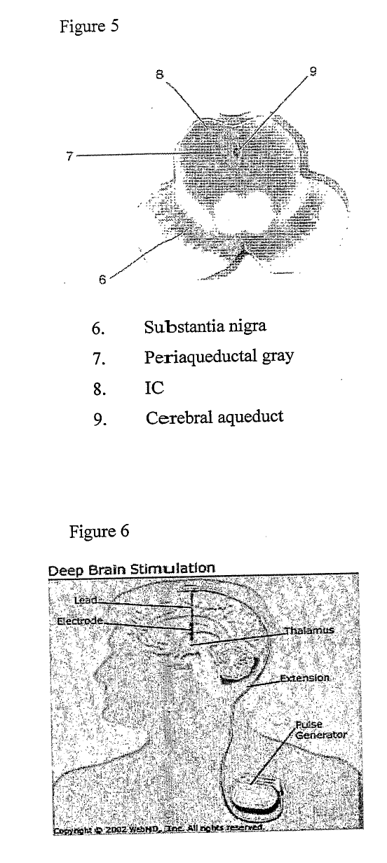

### DESCRIPTION OF THE PREFERRED EMBODIMENTS

The present invention is of an implantable electrode system which can be used to treat tinnitus and other neurological and otological disorders by placement in the IC and its adjacent region. The principles and operation of an implantable IC electrode system according to the present invention may be better understood with reference to the drawings and the accompanying description.

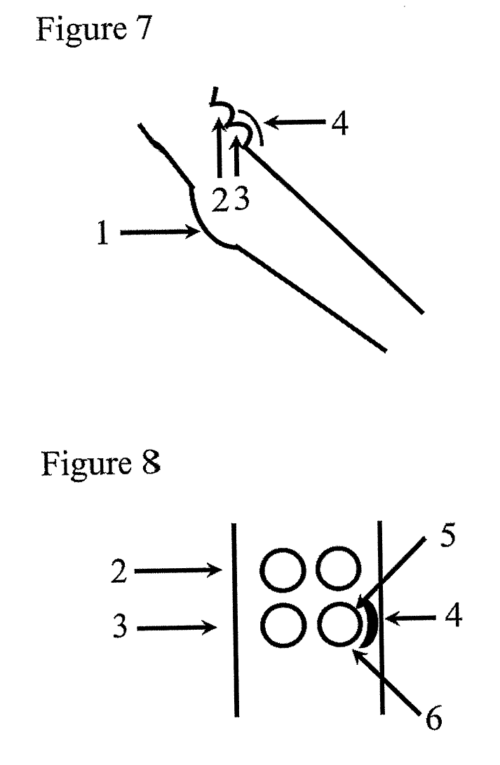

Referring now to the drawings, FIG. 7 illustrates the anatomy of the midbrain. Part 1 is the pons, Part 2 is the SC, Part 3 is the IC. The non-linear shape Part 4 is the implantable IC surface electrode. Its shape in the drawing indicates only that the shape of the electrode will match the shape of the IC.

FIG. 8 is a back view of the colliculi. By illustration, Part 2 is the SC and Part 3 is the IC. The electrode, Part 4 is positioned so that the electrode aligns with the frequency mapping of the IC, illustrated by Parts 5 and 6, referring to a possible continuum of frequencies to which the IC is sensitive. It may include other configurations such as right to left. The number of stimulating points is only for demonstrative purposes.

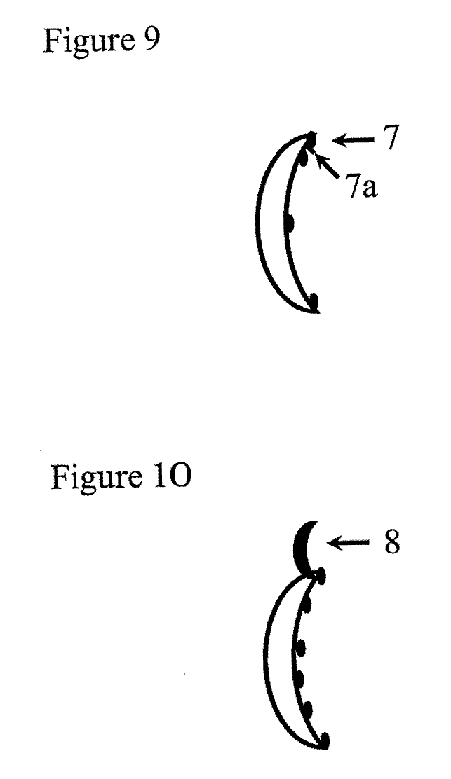

FIG. 9 illustrates the structure of a surface implant device that fits the frequency sensitivity of different parts of the IC using a plurality of electrode terminals (Part 7). The drawing does not necessarily portray the exact shape of the electrodes but is illustrative. In FIG. 9, the implant device will in one embodiment be a smooth surface, in which parts of the surface are small rounded stimulating surfaces (Part 7), with insulation (Part 7a) in between the stimulating surfaces. Adjacent to or on the electrode terminals, interaction with compounds provided from the electrode to the brain cavity is possible through a coating or cannula.

FIG. 10 illustrates one possible configuration of an anchoring piece (Part 8) attached to the implant device. It is possible to have other arms attached to the central electrode column with permanent or temporary attachment means. In this invention, the anchoring piece does not need to rely on anchoring within the brain tissue and subsequent scarring; rather, the anchoring occurs outside the brain tissue within the meningeal layers.

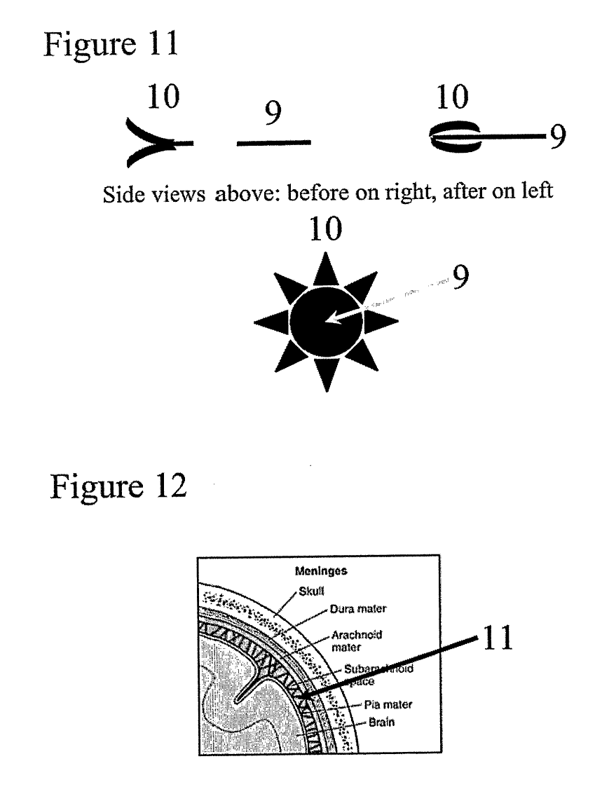

FIG. 11 illustrates one configuration of an implant device inserted in needle-like form with an internal needle-like shape (Part 9) and external petal-like structures that fan out after insertion (Part 10). FIG. 11 illustrates a side view and a cross-section. The number of petal-like structures in the figure and the shapes of the electrode are only for demonstration purposes.

FIG. 12 shows the layers of the meninges and the cranium. An implant device inserted at midline goes between the hemispheres and onto the IC without going through the cerebellum or the corpus callosum. In the ideal embodiment, the implant device will be placed in the space between the pia and the brain, but other configurations are possible, and the illustration shows an arrow to one possible area of placement.

As preparation for the methods that occur upon insertion, the patient undergoes a pre-operative assessment for implant device placement that includes but is not limited to history forms, analysis of current and previous patterns of hearing tests, and assessment of the following conditions associated with tinnitus, among others, at each stage of treatment: Phonal trauma, hyperacusis, head injury and its sequelae, Meniere disease, otologic hearing loss, sensorineural hearing loss, otosclerosis, ototoxic medication-induced tinnitus by drugs and toxins such as aspirin, non-steroidal anti-inflammatories, aminoglycosides, chloramphenicol, erythromycin, tetracycline, vancomycin, bleomycin, cisplatin, mechlorethamine, methotrexate, vincristine, furosemide, chloroquine, heavy metals, heterocyclic antidepressants, quinine, bumetanide, and ethacrinic acid, thyroid disorders, hyperlipidemia, vitamine B12 deficiency, multiple sclerosis, arteriovenous malformation, vascular tumors, palatomyoclonus, idiopathic stapedial muscle spasm, patulous Eustachian tube, Bell's palsy, stapedectomy, Ramsay Hunt syndrome, noise-induced hearing loss, recruitment, perilymphatic fistula, depression, migraine, Williams' syndrome, Addison's disease, Lyme diseases, genetic disorders, acoustic overstimulation, and acoustic neuroma.

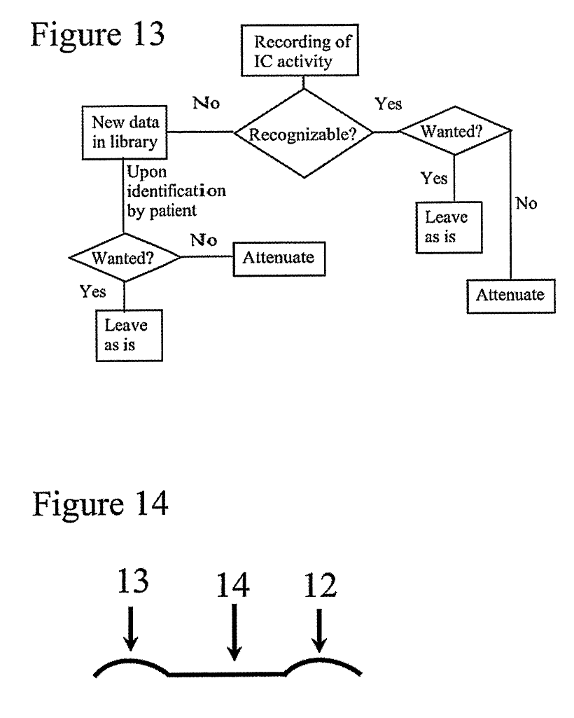

FIG. 13 is a flow chart that illustrates one possible embodiment of the feedback process for determining the correct application of stimulation parameters. It shows a a feedback process which will determine the stimulation parameters specific to each patient by recording and analyzing electrical activity by means of said electrode; recording the effect of stimuli at different locations within the electrode array; delivering stimuli to specific points in the inferior collicular and adjacent region; asking the patient about his/her experience with different stimulation parameters delivered to the IC region; inclusion and development of a library of stimulation parameters, measuring auditory, pain, and/or other neurological signals, with reception, storage and analysis of the stimulation parameters in the implanted system with calculation of subsequent stimulation parameters in conjunction with patient input; said implant system storing information on the ideal stimulation point, frequency, and intensity of the stimulation; providing the programmed options of restoration to initial configuration, and saving of multiple custom configurations with names for each setting in the memory of the apparatus, with the option to adjust one parameter of each titled setting at a time; providing an artificially intelligent computer system within the implantable system that will accept input from the patient when the tinnitus is greatest and simultaneously record brain potentials, and construct an algorithm for the automatic stimulation of the best possible parameters at the appropriate time.

The process of functional imaging to determine the placement of the implant device is by a combination of imaging techniques and auditory and sensory stimulation at different frequencies, electronically mapping to a computer system the functions found in the inferior collicular area, and placing and controlling the implant device in accordance with such mapping. The implantable system makes that process possible by providing a computer and remote control device that interacts with the stimulator and implant device.

FIG. 14 is a cross-section of an implant device configuration with a shape that approximates the external shape of the SC, PAG, and IC. Part number 12 represents a cap over the IC, Part number 13 represents a cap over the SC, and Part number 14 represents the section apposing the PAG. Each portion contains distinct electrode arrays. In this manner, electrical stimulation can be delivered as accurately as possible to the brain structures and the feedback system described for the inferior colliculus can be applied to other medical problems and the SC and PAG.

Whether the SC is stimulated from an IC electrode of FIG. 7 or the combined IC, PAG, and SC electrode of FIG. 14, the stimulation of the SC will occur as part of a method of treating partial hearing loss by recording stimulation to the region under the influence of various stimuli, thereby assessing which areas and parameters of electrode stimulation result in muscle movements that incline the patient towards the wanted auditory stimulus.

While the invention has been described with respect to a limited number of embodiments, it will be appreciated that many variations, modifications and other applications of the invention may be made.

1-59. (canceled)

60. A method for treating tinnitus of a subject, comprising:
coupling an implant device to an inferior colliculus of the subject, the implant device including an electrode; and
treating the tinnitus by driving a current into a vicinity of the inferior colliculus via the electrode.

61. The method according to claim 60, wherein treating the tinnitus comprises inducing a change in an auditory pathway of the subject, by driving the current into the vicinity of the inferior colliculus.

62. The method according to claim 60, wherein coupling the implant device to the inferior colliculus comprises deploying a majority of the implant device outside of brain tissue.

63. The method according to claim 60, wherein coupling the implant device to the inferior colliculus comprises implanting the implant device so that it substantially apposes at least the inferior colliculus.

64. The method according to claim 60, wherein driving the current comprises driving the current into a somatotropic area in the vicinity of the inferior colliculus.

65. The method according to claim 60, wherein driving the current comprises performing anodal blocking.

66. The method according to claim 60, wherein the subject has a first ear and a second ear, wherein the method comprises identifying the first ear as having greater tinnitus than the second ear, and wherein coupling the implant device to the inferior colliculus comprises coupling the implant device contralaterally to the first ear.

67. The method according to claim 60, comprising determining whether the tinnitus is bilateral, and wherein coupling the implant device to the inferior colliculus comprises coupling the implant device to a left inferior colliculus of the subject, responsively to determining that the tinnitus is bilateral.

68. Apparatus for treating tinnitus of a subject, comprising:
an implant device comprising an electrode and configured to be disposed in a vicinity of an inferior colliculus of the subject; and
a control unit which is configured to treat the tinnitus by driving current into the vicinity of the inferior colliculus via the electrode.

69. The apparatus according to claim 68, wherein the electrode comprises a plurality of electrodes.

70. The apparatus according to claim 68, wherein the electrode has a characteristic length that is less than 0.25 times a characteristic length of the implant device.

71. The apparatus according to claim 68, wherein the implant device is substantially planar, and a plane of the implant device is configured to be implanted substantially parallel to a plane of a brain surface of the subject.

72. The apparatus according to claim 68, wherein the implant device is configured to appose the inferior colliculus.

73. The apparatus according to claim 68, wherein the implant device is configured to cover at least the inferior colliculus.

74. The apparatus according to claim 68, wherein a ratio between a characteristic dimension of the implant device and a characteristic dimension of a brain of the subject is less than 0.4.

75. The apparatus according to claim 68, wherein the electrode is configured to deliver current into a somatotropic area in the vicinity of the inferior colliculus.

76. The apparatus according to claim 68, wherein the implant device comprises an arm configured to attach to a cranial structure.

77. The apparatus according to claim 68, wherein the implant device is configured to change shape from a needle-like shape to a fan-like shape.

78. The apparatus according to claim 68, wherein the implant device comprises an anodal blocking element.

79. The apparatus according to claim 68, wherein the implant device is coated with an agent which is configured to interact with brain tissue.

80. The apparatus according to claim 68, wherein the implant device comprises a conduit which is configured to facilitate delivery, to brain tissue of the subject, of an agent configured to interact with the brain tissue.

81. The apparatus according to claim 68, wherein the control unit is configured to receive an input and to control the driving of current to the electrodes in response thereto.

82. The apparatus according to claim 81, wherein the input includes feedback from the electrode, and wherein the control unit is configured to receive the feedback.

83. The apparatus according to claim 81, wherein the input includes an input from a human, and wherein the control unit is configured to receive the input from the human.

84. The apparatus according to claim 81, comprising a computer, wherein the input includes an input from the computer, and wherein the computer is configured to store a treatment parameter and to send the input in response treatment parameter.

85. A method for treating a condition of a subject, the condition selected from the group consisting of: tinnitus and pain, the method comprising:
implanting an electrode on a surface of a brain of the subject in a vicinity of an inferior colliculus of the subject; and
treating the condition by driving a current into the vicinity of the inferior colliculus via the electrode.

86. Apparatus for treating pain in a subject, comprising:
a planar electrode, configured to be deployed on a surface of a brain of the subject in a vicinity of an inferior colliculus of the subject; and
a control unit, configured to drive the planar electrode to apply a current to the vicinity of the inferior colliculus.

87. A method for implanting a device in a brain of a subject, comprising:
coupling an anchoring element to the device; and
anchoring the anchoring element to a site selected from the group consisting of: a meningeal layer of the subject, and a skull of the subject.

88. The method according to claim 87, wherein the anchoring element includes a first portion having a needle shape, and a second portion configured to fan out, and wherein anchoring the anchoring element comprises facilitating the second portion to fan out.
